(19) United States
(12) Patent Application Publication
(10) Pub. No.: US 2009/0227669 A1
(43) Pub. Date: Sep. 10, 2009

(54) Compositions and Methods for Perioperative Bladder Instillation

(75) Inventors: J. Karl Kemberling, Eden Prairie, MN (US); Steven H. Selman, Toledo, OH (US)

Correspondence Address: FISH & ASSOCIATES, PC;ROBERT D. FISH, 2603 Main Street, Suite 1000, Irvine, CA, 92614-6232, US

(73) Assignee: THE UNIVERSITY OF TOLEDO, Toledo, OH (US)

(21) Appl. No.: 12/423,205

(22) Filed: Apr. 14, 2009

Related U.S. Application Data
(63) Continuation-in-part of application No. 11/040,908, filed on Jan. 21, 2005.
(60) Provisional application No. 60/538,699, filed on Jan. 23, 2004.
(60) Provisional application No. 61/045,452, filed on Apr. 16, 2008.

(51) Int. Cl. A61K 31/352; A61P 41/00

(52) U.S. Cl. 514/456

(57) ABSTRACT

Polyphenol- and especially catechin-containing compositions (e.g., Polyphenon E) are instilled in the bladder of a subject that previously underwent surgical resection or removal of bladder cancer to reduce cancer growth and/or recurrence.

This application is a continuation-in-part application of our copending U.S. application with the Ser. No. 11/040,908, filed Jan. 21, 2005 which claims priority to U.S. provisional 60/538,699, filed Jan. 23, 2004. This application also claims priority to our copending U.S. provisional application with the Ser. No. 61/045,452, filed Apr. 16, 2008, all of which are incorporated by reference herein in their entirety.

### FIELD OF THE INVENTION

The field of the invention is perioperative, and particularly co- and/or postoperative treatment of a patient undergoing surgery for bladder cancer wherein the treatment comprises bladder instillation of polyphenols and especially catechin-containing compositions.

### BACKGROUND

Bladder cancer is a relatively common malignancy, and various pharmacological and/or naturopathic treatment modalities have been proposed. For example, U.S. Pat. Nos. 6,410,061, 6,410,052, 6,428,818, and 6,652,890 describe methods for treating cancer or solid tumors with EGCG, which is typically orally administered or administered by injection. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

Similarly, DJang et al. describe in U.S. Pat. No. 6,168,795 an herbal composition (including extracts of Gynostemma pentaphyllum, Crataegus pinnatifida, and Camellia sinensis) that was thought to be effective as a stand-alone modality in non-surgical treatment of various established cancers, including bladder carcinoma, possibly due to the saponins and flavonoids and polyphenols present in the extract of Crataegus pinnatifida, and the polyphenols present in the extract of Camellia sinensis. Similarly, Sato et al. described (International Journal of Urology (2003) 10, 160-166; International Journal of Urology (1999) 6, 93-99) oral administration of green tea as non-surgical therapeutic modality against bladder tumors induced in rats by the carcinogen, N-butyl-N-(4-hydroxybutyl)-nitrosamine (BBN). However, it should be noted that orally administered green tea catechins are subject to substantial metabolic modification and fecal excretion. Therefore, only a minute fraction of catechins and catechin metabolites will be excreted via the renal route (e.g., 0.03-0.59% of total administered EGCG along with at least five metabolites is found in urine; see Suganuma et al. in Carcinogenesis vol. 19 no. 10 pp. 1771-1776. 1995). Therefore, any direct anti-carcinogenic effect of catechins in the bladder can be ruled out.

Consequently, treatment of established cancer or solid tumors with orally administered or systemically injected EGCG is typically insufficient. Therefore, surgery is required in most cases to reduce or entirely eliminate the cancer burden. Surgery may further be accompanied by one or more additional treatment options, chemotherapy and/or radiation.

Unfortunately, recurrence of bladder cancer after transurethral resection is relatively high (>60% in 7 years) and is at least in some instances thought to correlate with postoperative tumor cell re-implantation. Various intravesical agents have been utilized in an attempt to decrease the rate of recurrence. However, currently used agents exhibit only moderate success rates and often undesirable side effects. For example, Kiyohara et al. report numerous side effects with postoperative chemotherapy using cyclophosphamide, cis-platinum, and adriamycin (see e.g., Cancer Chemotherapy and Pharmacology, Volume 20, Supplement 1/August, 1987). In an attempt to use non-cytostatic/cytotoxic drugs, Oladele et al report in U.S. Pat. No. 6,037,332 instillation with a dextran sulfate solution to prevent growth of bladder carcinoma cells, presumably via inhibitory effects on binding of insulin-like growth factor to bladder tumor cells. While such compounds are generally nontoxic or have a relatively low toxicity, relatively high volume and concentrations need to be applied. Moreover, the results reported by Oladele were only based on a N-methyl-N-nitrosurea (MNU)-induced rat bladder transitional cell carcinoma model and have not been reproduced in human.

Therefore, while numerous treatment options are known in the art, they are often not well tolerated and tend to produce new difficulties. Moreover, Bono et al reported (Cancer Chemother Pharmacal (1994) 35 (Suppl): S 101-S 109) that it would be doubtful that post-operative bladder instillation with various chemotherapeutic drugs would have any effect on long-term recurrence rates, progressions, or disease-specific mortality. Thus, and not surprisingly, Bono reported that no uniform consensus had been reached on the basic indications, the treatment duration, the timing for the start, the choice of drug, or the schedule for bladder instillation.

Remarkably, despite the wealth of literature on green tea and its use in chemoprevention and treatment of cancer, there are no known reports on the efficacy of polyphenolic compounds, catechins, and in particular EGCG, as intravesical therapeutic agent, especially in the context of perioperative or postoperative treatment of bladder cancer. Thus, there is still a need for improved perioperative or postoperative treatments, especially with compositions and methods that are well tolerated and non-toxic.

### SUMMARY OF THE INVENTION

The present invention is directed to compositions and methods of treatment of a person that that is scheduled for or underwent surgery for bladder cancer in which a polyphenol-, and most preferably a catechin-containing composition is instilled into the bladder of the person to reduce recurrence of bladder cancer after surgery.

In one aspect of the inventive subject matter, a method of perioperatively treating a person undergoing surgery for bladder cancer will include a step of instilling a catechin-containing composition into the bladder of the subject at a concentration effective to reduce bladder cancer recurrence after surgery. Where desired, the catechin-containing composition may further comprise an additional therapeutic agent. Most preferably, instillation is preoperatively and/or postoperatively and/or during surgery. Additionally, contemplated treatment may be accompanied by perioperatively providing an oral supplement that includes a catechin-containing composition.

It is further contemplated that the catechin-containing composition comprises a plurality of chemically distinct catechins at a concentration of at least 100 microM, more preferably at least 200 microM, and most preferably at least 400 microM. While numerous dwell times for instillation are deemed suitable, it is further preferred that instillation is performed with a dwell time of at least 60 minutes, and most preferably at least 120 minutes. IT is further contemplated that catechin-containing composition may be formulated as sustained release formulation or as a solution.

Consequently, in another aspect of the inventive subject matter, a kit may include a catechin-containing composition that is formulated for instillation into a bladder of a person, and a catheter suitable for administration of the catechin-containing composition. Especially suitable catechin-containing composition will comprise EGCG at a concentration of at least 100 microM or will comprise a mixture of chemically distinct catechins at a total concentration of at least 100 microM. Where desired, the catechin-containing composition may be formulated as a sustained release formulation.

In a further aspect of the inventive subject matter, a controlled release preparation is contemplated that is formulated for placement into a bladder through the urethra of a person wherein the preparation comprises a catechin-containing composition as a pharmaceutically active ingredient and a pharmaceutically acceptable carrier, and wherein the carrier is capable of sustained delivery of the catechin within the bladder. For example, the carrier may be bio-erodable over a desired period of time within the bladder. Moreover, it is contemplated that the preparation may be part of a kit that further includes a catheter.

Various objects, features, aspects and advantages of the present invention will become more apparent from the following detailed description of preferred embodiments of the invention.

### BRIEF DESCRIPTION OF THE DRAWING

FIG. 1 depicts exemplary photomicrographs of a typical gross appearance of control bladder with tumors (0 μM PE, 0 dwell time) and the corresponding H&E stained section.

FIG. 2 depicts exemplary photomicrographs of a typical gross appearance of a tumor free bladder (400 μM PE, 120 min dwell time) and the corresponding H&E stained section.

FIG. 3 depicts exemplary photomicrographs of a gross appearance of a bladder with a questionable area (100 μM PE, 120 min dwell time) and the corresponding H&E stained section showing small tumor growth.

FIG. 4 depicts exemplary photomicrographs of a gross appearance of a bladder with a questionable area (100 μM PE, 120 min dwell time) and the corresponding H&E stained section showing no tumor growth.

FIG. 5 is an experimental flow chart for an exemplary treatment according to the inventive subject matter.

### DETAILED DESCRIPTION

The present invention is directed to various compositions and methods of perioperative, and especially co- and postoperative treatment of a patient that previously underwent surgical resection or removal of bladder cancer. In contemplated methods, a polyphenol and/or catechin-containing composition is instilled into the bladder, typically in an aqueous solvent in which the polyphenol, and/or the catechin (and particularly EGCG) is present at a concentration of at least 50 microM, and more typically at least 100 microM for a dwell time of at least 30 minutes, and more preferably at least 60 minutes.

For example, an aqueous sterile solution comprising EGCG at a concentration of between about 200-400 microM is used to rinse the bladder during bladder cancer resection and the same type of solution is instilled after surgery via transurethral catheter, typically for a dwell time of between 30 and 120 minutes. Post-surgical instillation is typically performed on a schedule that requires instillation between once daily and once monthly (most typically once to twice a week) over a period of several weeks to months using a dwell time of about 60-90 minutes. Where desired, a sustained release preparation may also be introduced during or after surgery to provide a continuous exposure of the bladder tissue to the EGCG (e.g., using a bio-erodable carrier that is formulated such that during or after the release of the drug there is no blockage of the urinary tract while the sustained release preparation is within the bladder).

As used herein, the term “perioperative” refers to a period of time before, during, and after surgery, wherein the time ‘before surgery’ begins when a physician orders or recommends surgery to remove or reduce bladder cancer and ends at the beginning of surgery (typically when anesthesia commences and/or when a transurethral cystoscope or resectoscope is inserted), and wherein the time ‘after surgery’ begins when the anesthesia is terminated and/or when the transurethral cystoscope or resectoscope is removed from the patient. Therefore, as used herein the term “postoperative” refers to a period when the transurethral cystoscope or resectoscope is removed from the patient, and the term “co-operative” refers to the period of surgery. As still further used herein, the terms “instillation” and “intravesical administration” are used interchangeably and refer to administration of a composition into the bladder of a patient using a route other than renal excretion into the bladder. For example, the terms “instillation” and “intravesical administration” include transurethral administration (typically using a catheter) to the bladder, direct injection into the bladder, and administration into the bladder via a catheter passing across the abdominal wall (e.g., via suprapubic catheter). Therefore, the terms “instillation” and “intravesical administration” expressly exclude delivery of urine from the kidney(s) to the bladder via the ureter.

It is generally contemplated that the compounds suitable for use in conjunction with the teachings presented herein are polyphenolic compounds, and particularly tannins, lignins, and flavonoids. However, it is especially preferred that the polyphenolic compounds are selected flavonoids (e.g., flavonols, flavones, catechins, flavanones, anthocyanidins, and isoflavonoids), all of which may be further modified by modification with a glycosidic moiety and/or a phenolic moiety (e.g. gallate). Thus, especially preferred compounds will be those that naturally occur in plants. Furthermore, and especially where such compounds are obtained by extraction from one or more plant materials, it should be appreciated that the compounds may be employed as single chemical entities (e.g., isolated compound purity of at least 80%, more typically at least 90%) or as mixture of multiple and chemically distinct compounds. Examples for isolated compounds especially include catechin, epicatechin, epigallocatechin, and epigallocatechin gallate, all of which may be enantiomerically pure or in any isomeric mixture. Examples for mixtures of compounds especially include alcoholic extracts of fruit and/or leaves from various plants, and particularly from green tea (e.g., Polyphenon E).

Further contemplated compounds include various synthetic forms of naturally occurring compounds as well as chemical modifications thereof (e.g., esters, thioesters, amides, etc.). For example, chemically modified polyphenols and especially catechins suitable for use herein include those described in U.S. Pat. Nos. 4,237,162, 4,617,296, U.S. Pat. App. No. 2002/0151582, WO 2004/052873, and WO 99/22728, all of which are incorporated by reference herein. Still further, also contemplated suitable for use herein are all prodrug forms and metabolites of the polyphenolic and especially catechin compounds described herein.

Thus, it should be appreciated that suitable compositions may vary considerably and that compositions according to the inventive subject matter will typically include complex mixtures of natural origin, synthetic complex preparations. Most preferably, however, the compositions will include between one and three at least partially isolated catechins (e.g., EGCG, EGC, and EC). With respect to the concentration of the polyphenol in the compositions that are instilled into the bladder it is generally preferred that the concentrations of the compounds will typically be at least 50 microM (e.g., between 50 and 100 microM, in less preferred cases between 1-50 microM), more typically at least 100 microM (e.g., between 100 and 200 microM), even more typically at least 200 microM (e.g., between 200 and 400 microM), and most typically at least 400 microM (e.g., between 400 and 800 microM, and even higher).

It should be noted that there are numerous pharmaceutically acceptable carriers for instillation known in the art, and all of them are deemed appropriate for use herein. However, it is particularly preferred that the carrier is an aqueous carrier (e.g., comprises at least 50 wt % water as a solvent). Still further preferred solvents and co-solvents include those that are pharmaceutically acceptable, and especially, N-methylpyrrolidone (NMP), acetone, anisole, dimethylacetamide (DMA), dimethylformamide (DMF), pyrrolidone, tetrahydrofuran (THF), benzyl benzoate, an alkyl lactate, a glycofurol, a short chain alcohol, a polyethylene glycol (PEG), propylene glycol, polyoxyl castor oil, glycerol, almond oil, cyclodextrin, a polyvinyl alcohol, and propylene carbonate

Thus, particularly preferred instillation compositions will comprise an aqueous and pharmaceutically acceptable carrier with at least one catechin at a concentration of at least 50 microM, and all known carriers for instillation solutions are deemed suitable for use herein. Of course, it should be recognized that additional compounds may be added to the instillation formulation, and especially suitable compounds include known therapeutic compounds for treatment of bladder cancer (e.g., cytostatic and/or cytotoxic agents), anti-oxidants, and various radionuclides.

Moreover, it should be noted that the compounds suitable for use herein may also be formulated into a controlled release formulation that is deposited into the bladder (typically via transurethral route). In such case, it is generally preferred that an average concentration of contemplated compounds of at least 50 microM, and more preferably at least 100 microM is achieved, and/or that the bladder is continuously exposed to the compounds over a period of at least 12 hours, more preferably at least 24 hours, even more preferably at least 3 days, and most preferably at least 7 days. There are numerous known controlled release formulation for bladder instillation in the art, and all of those are deemed suitable for use herein. For example, suitable controlled release formulations include those described in U.S. Pat. App. No. 2001/0019719, 2004/0197397, and U.S. Pat. Nos. 7,361,368, and 6,894,071.

With respect to timing and dwell time of instillation it is contemplated that the timing of instillation may be varied substantially. For example, it is contemplated that instillation may be performed prior to surgery, most typically within 0 to 12 hours, less typically within 12-24 hours, and in some cases even longer. Similarly, the bladder may be rinsed during surgery as adjunct or even replacement to saline rinses. Post-operative instillation may be performed at various times, typically at the conclusion of the surgery, followed by one or more post-operative instillations that are between several hours and several days after conclusion of surgery. Therefore, it should be appreciated that instillation with contemplated compounds may be performed perioperatively and more typically postoperatively at one or more occasions using identical or different catechin (or other polyphenol) concentrations. Similarly, it should be appreciated that the dwell time may vary considerably, and suitable dwell times may be between single rinsing events and several hours. However, it is generally preferred that the dwell time is between about 30 minutes and 300 minutes, and most preferably between 60 minutes and three hours. Of course, it should be noted that where contemplated compositions are formulated as controlled release formulations (e.g., using a bio-erodable or biodegradable carrier), the dwell time will be between several hours and several days (if not even weeks).

In still further contemplated aspects, it should be appreciated that treatment of bladder cancer may include oral supplementation with catechin (or other polyphenolic) compositions, typically starting as pre-operative treatment before surgery and lasting several weeks, months, and even years after surgery. For example, especially preferred oral supplementation with catechin compositions will comprise administration of a complex catechin composition (e.g., Polyphenon E or other green tea extract, or isolated catechins such as EGCG) at a daily dosage range between 50 mg and 2000 mg, and more preferably between 200 mg and 1200 mg. Thus, and in light of the experimental data below, the inventor contemplates compositions and methods for peri-operative treatment of a person that underwent surgery for bladder cancer in which a catechin containing composition in instilled into the bladder. Most preferably, the instillation is co- and postoperatively and under a protocol effective to reduce recurrence of bladder cancer.

In yet further less preferred aspects, instillation of polyphenols, and in particular catechin mixtures or isolated catechins (e.g., EGCG) can be used alone or in combination with other known therapeutic agents and methods of treatment for the treatment of bladder cancer without surgery in a manner and using compositions as provided above. For example, the intravesical administration of polyphenols, and in particular EGCG, can be used before during and after administration of other therapeutic agents and methods including, radiation and chemotherapy.

### EXAMPLES

Single Polyphenol (EGCG): Fisher 344 rats were maintained under guidelines of the Medical College of Ohio Institutional Animal Care and Use Committee. A combination ketamine (80 mg/kg)/xylazine (12 mg/kg) intraperitoneal injection was used to anesthetize the rats prior to surgery. The abdomen was shaved and prepped with an iodine solution under sterile conditions. Oxytetracycline 20 mg was injected subcutaneously for antibacterial prophylaxis. Under 4× microscopic magnification, a 1.5 cm midline incision exposed the bladder. A sterile, 22 gauge Angiocath cannula was then advanced transurethrally. A blunt-tipped 24 gauge copper wire was then advanced through the catheter and a 6 mV current was used to cauterize the bladder wall creating a small, white puckered area. The skin and muscular layers of the abdominal wall were closed with a running 4-0 silk suture.

AY-27 cells suspended in 0.25 cc of serum free RPMI-1640 medium were instilled transurethrally into 40 rats. The catheters were then capped and the tumor cells were allowed to dwell inside the bladder for 30 minutes. After tumor cell instillation, twelve rats (Group I) had two, separately hourly instillations of culture medium. Twenty-eight rats (Group II) were treated with a freshly prepared solution of 0.25 cc, 200 microM EGCG solution in RPLMI-1640 medium for 2 separate dwell times of one hour.

At the end of two hours the catheters were drained. All rats survived and were transferred to the animal care facility for recovery. The rats were euthanized three weeks later. Bladders were removed and placed in 10% phosphate buffered formalin solution and allowed to fix for several hours. Bladders were hemisected with a razor, examined under 4× magnification and sent for histopathologic sectioning. Statistical analysis was performed using Sigma Stat 2.03 software.

Group I controls (n=12) demonstrated 100% tumor growth rate. Group II (n=28), EGCG treated rats had a tumor growth rate of 36% (10 of 28 rats). Statistical significance was achieved with a p value of 0.001 using the Fisher exact test. Numerous theories have evolved in an attempt to explain the action of EGCG, but our results for the AY-27 cell line do not indicate that apoptosis is the primary mechanism of action. The AY-27 cells only revealed a weak banding pattern on DNA ladder assay, suggesting that other mechanisms may be involved in their death. No cellular survival was seen after two hours of treatment at all concentrations of EGCG.

Thus, it should be appreciated that epigallocatechin-gallate (EGCG) inhibits cellular growth of the AY-27 rat transitional cancer cell line. Instillation of intravesical EGCG at 200 microM for 2 hours appear to significantly reduce the incidence of bladder tumor implantation in the Fisher 344 rat model.

Polyphenol mixture (Polyphenon E): Fisher 344 rats were maintained under guidelines of the Medical College of Ohio Institutional Animal Care and Use Committee. A combination ketamine (80 mg/kg)/xylazine (12 mg/kg) intraperitoneal injection was used to anesthetize the rats prior to surgery. The abdomen was shaved and prepped with an iodine solution under sterile conditions. Oxytetracycline 20 mg was injected subcutaneously for antibacterial prophylaxis. Under 4× microscopic magnification, a 1.5 cm midline incision exposed the bladder. A sterile, 22 gauge Angiocath cannula was then advanced transurethrally. A blunt-tipped 24 gauge copper wire was then advanced through the catheter and a 6 mV current was used to cauterize the bladder wall creating a small, white puckered area. The skin and muscular layers of the abdominal wall were closed with a running 4-0 silk suture.

AY-27 cells suspended in 0.25 cc of serum free RPMI-1640 medium were instilled transurethrally into 10 rats. The catheters were then capped and the tumor cells were allowed to dwell inside the bladder for 30 minutes. After tumor cell instillation, five rats (Group I) had two, separately 30 minute instillations of culture medium. Five rats (Group II) were treated with a freshly prepared solution of 0.5 cc, 200 microM Polyphenon E solution in RPLMI-1640 medium for 2 separate dwell times of 30 minutes. At the end of one hour the catheters were drained. All rats survived and were transferred to the animal care facility for recovery. The rats were euthanized three weeks later. Bladders were removed and placed in 10% phosphate buffered formalin solution and allowed to fix for several hours. Bladders were hemisected with a razor, examined under 4× magnification and sent for histopathologic sectioning. Statistical analysis was performed using Sigma Stat 2.03 software.

Group I controls (n=5) demonstrated 100% tumor growth rate. Group II (n=5), Polyphenon E treated rats had a tumor growth rate of 20%. Statistical significance was achieved with a p value of 0.001 using the Fisher exact test.

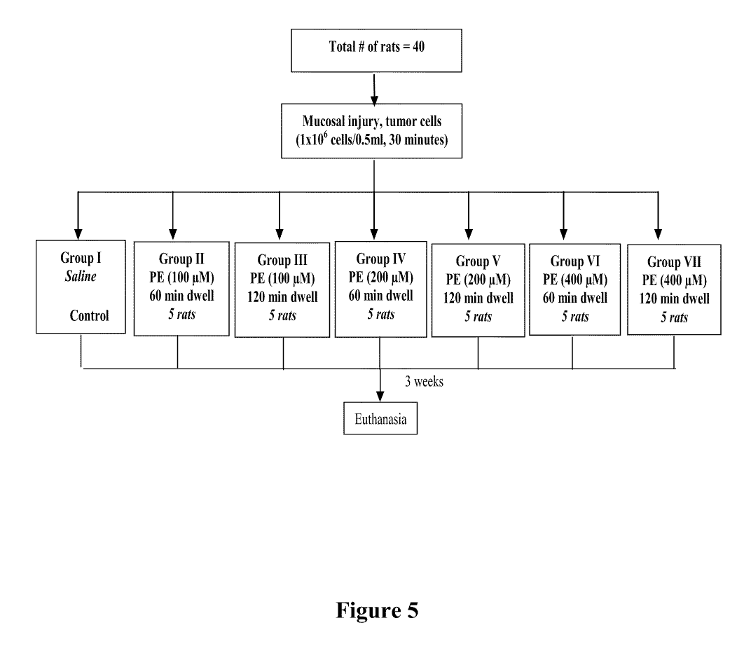

Variable Dosages/Times: To investigate which dosage and/or dwelling time may be most effective, the concentration of P/E (0, 100, 200 400 μM) was varied and three different dwell times were used (0, 60, and 120 minutes). The experimental procedure of the above examples was used in this study and is schematically outlines in FIG. 5. Briefly, for the group of animals that had a dwell time 120 minutes, the bladders were gently emptied after the first 60 minutes and re-filled with fresh treatment solution for the next 60 minutes. The pH of all treatment solutions was adjusted to 5.0 prior to instillation into the bladder. The volume of instilled cells and treatment solutions was 0.5 ml. The number of cells instilled into the bladder was 1×106.

Animals were euthanized 3 weeks after treatment. The bladders were filled with 10% buffered formalin, tied off and removed. After the bladders were allowed to fix for 24 hrs they were cut in half longitudinally. The bladders were examined under a microscope at 10× power and photographed. Any tumor growth or suspicious areas were noted and marked with India ink and submitted for histology. A Pathologist examined the histology and the observations were recorded. (See Table 1 below)

| Treatment Group | | | Tumor Growth | | |
|---|---|---|---|---|---|
| PE Conc | Dwell Time | | Gross | Histological | Tumor |
| (uM) | (min) | Rat # | Observation | Observation | Ratio |
| 0 | 60 | 468 | + | + | 10/10 |
| | | 469 | + | + | |
| | | 492 | + | + | |
| | | 493 | + | + | |
| | | 301 | + | + | |
| | | 315 | + | + | |
| | | 316 | + | + | |
| | | 331 | + | + | |
| | | 339 | + | + | |
| | | 340 | + | + | |
| 0 | 120 | 476 | + | + | 10/10 |
| | | 477 | + | + | |
| | | 484 | + | + | |
| | | 485 | + | + | |
| | | 302 | + | + | |
| | | 323 | + | + | |
| | | 324 | + | + | |
| | | 332 | + | + | |
| | | 347 | + | + | |
| | | 348 | + | + | |
| 100 | 60 | 470 | + | + | 7/10 |
| | | 471 | + | + | |
| | | 494 | − | − | |
| | | 495 | − | − | |
| | | 303 | + | + | |
| | | 313 | + | + | |
| | | 314 | − | − | |
| | | 329 | + | + | |
| | | 374 | + | + | |
| | | 338 | + | + | |
| 100 | 120 | 478 | ? | − | 5/10 |
| | | 479 | − | − | |
| | | 486 | − | − | |
| | | 487 | ? | + | |
| | | 304 | ? | − | |
| | | 321 | ? | − | |
| | | 322 | + | + | |
| | | 330 | + | + | |
| | | 345 | + | + | |
| | | 346 | + | + | |
| 200 | 60 | 472 | − | − | 4/10 |
| | | 473 | + | + | |
| | | 496 | ? | − | |
| | | 497 | ? | − | |
| | | 305 | ? | − | |
| | | 311 | − | | |
| | | 312 | + | + | |
| | | 327 | ? | + | |
| | | 335 | + | + | |
| | | 336 | − | − | |
| 200 | 120 | 480 | − | − | 2/10 |
| | | 481 | − | − | |
| | | 488 | − | − | |
| | | 489 | ? | − | |
| | | 306 | − | − | |
| | | 319 | + | + | |
| | | 320 | ? | + | |
| | | 328 | ? | − | |
| | | 343 | − | − | |
| | | 344 | ? | − | |
| 400 | 60 | 474 | − | − | 2/10 |
| | | 475 | ? | − | |
| | | 498 | − | − | |
| | | 499 | ? | − | |
| | | 307 | + | + | |
| | | 309 | ? | − | |
| | | 310 | ? | − | |
| | | 325 | − | − | |
| | | 333 | − | − | |
| | | 334 | ? | + | |
| 400 | 120 | 482 | − | − | 0/10 |
| | | 483 | − | − | |
| | | 490 | − | − | |
| | | 491 | − | − | |
| | | 308 | − | − | |
| | | 317 | − | − | |
| | | 318 | − | − | |
| | | 326 | − | − | |
| | | 341 | − | − | |
| | | 342 | − | − | |

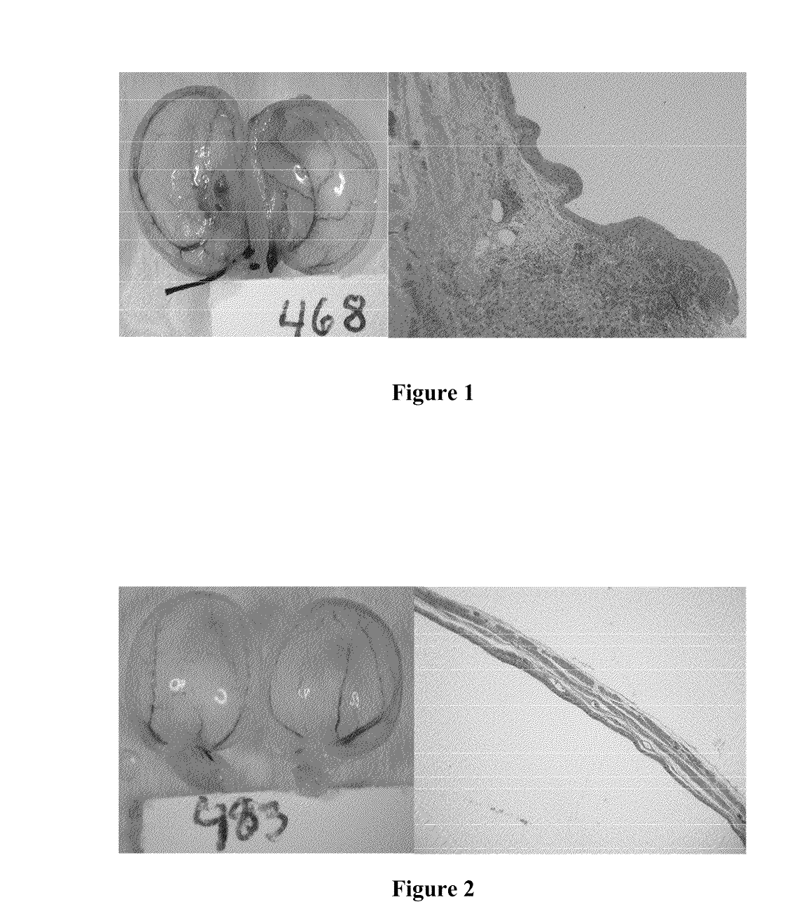

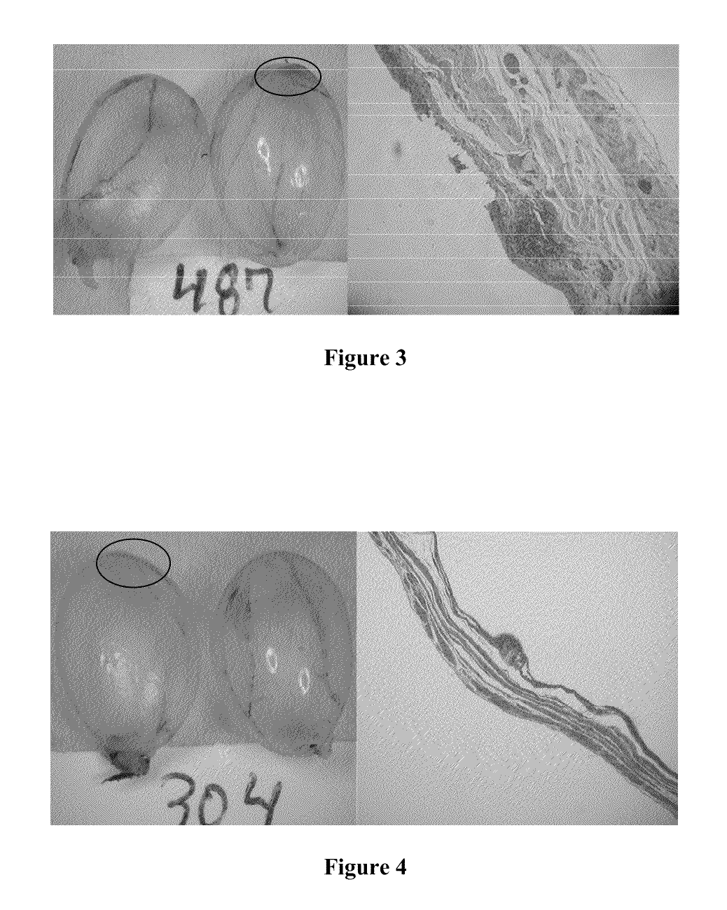

Exemplary bladder morphology and histology findings are depicted in FIGS. 1 to 4. Tumor growth ratios based on gross and histological evidence for each group are recorded in Table 2 below.

| PE Conc | Dwell Time | Tumor Growth |
|---|---|---|
| (μM) | (minutes) | Ratio |
| 0 | 60 | 10/10 |
| 0 | 120 | 10/10 |
| 100 | 60 | 7/10 |
| 100 | 120 | 5/10 |
| 200 | 60 | 4/10 |
| 200 | 120 | 2/10 |
| 400 | 60 | 2/10 |
| 400 | 120 | 0/10 |

Based on the above, the inventors also contemplate pretreatment of the bladder mucosa prior to bladder surgery. Most typically, concentrations will be as outlined above, but higher or lower concentrations and dwell times are also deemed suitable. In yet additional aspects of the inventive subject matter, it is also contemplated that the compositions according to the inventive subject matter may be effective as a non-surgical treatment after establishment of early stage bladder cancer.

Thus, specific embodiments and applications of perioperative instillation with polyphenol and especially catechin-containing compositions have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of perioperatively treating a person undergoing surgery for bladder cancer, comprising a step of instilling a catechin-containing composition into the bladder of the subject at a concentration effective to reduce bladder cancer recurrence after surgery.

2. The method of claim 1 wherein instillation is preoperatively.

3. The method of claim 1 wherein instillation is at least one of postoperatively and during surgery.

4. The method of claim 1 wherein the catechin-containing composition comprises a plurality of chemically distinct catechins.

5. The method of claim 1 wherein the concentration is at least 100 microM.

6. The method of claim 1 wherein the concentration is at least 200 microM.

7. The method of claim 1 wherein the concentration is at least 400 microM.

8. The method of claim 1 wherein the instillation is performed with a dwell time of at least 60 minutes.

9. The method of claim 1 wherein the instillation is performed with a dwell time of at least 120 minutes.

10. The method of claim 1 wherein the catechin-containing composition further comprises an additional therapeutic agent.

11. The method of claim 1 further comprising a step of perioperatively providing an oral supplement to the person, wherein the supplement comprises a catechin-containing composition.

12. The method of claim 1 wherein the catechin-containing composition is formulated as a sustained release formulation.

13. A kit comprising a catechin-containing composition formulated for instillation into a bladder of a person, and a catheter suitable for administration of the catechin-containing composition.

14. The kit of claim 13 wherein the catechin-containing composition comprises EGCG at a concentration of at least 100 microM.

15. The kit of claim 13 wherein the catechin-containing composition comprises a mixture of chemically distinct catechins at a total concentration of at least 100 microM.

16. The kit of claim 13 wherein the catechin-containing composition is formulated as a sustained release formulation.

17. The kit of claim 13 wherein the catheter is a urethral or suprapubic catheter.

18. A controlled release preparation that is formulated for placement into a bladder through the urethra of a person comprising a catechin-containing composition as a pharmaceutically active agent and a pharmaceutically acceptable carrier, wherein the carrier is capable of sustained delivery of the catechin within the bladder.

19. The controlled release preparation of claim 18, wherein the pharmaceutically acceptable carrier is bio-erodable over a desired period of time within the bladder.

20. The controlled release preparation of claim 18 wherein the preparation is in a kit that further comprises a catheter.
